SEQUENCE LISTING (6 sequences)

SEQ ID NO: 1 - DNA, 15 bp, Artificial Sequence
  Synthetic oligonucleotide providing protection from F. tularensis
gctagacgttagcgt

SEQ ID NO: 2 - DNA, 10 bp, Artificial Sequence
  Synthetic oligonucleotide providing protection from Francisella tularensis
tcaacgttga

SEQ ID NO: 3 - DNA, 33 bp, Artificial Sequence
  oligonucleotide PCR primer
acaagtctcgagaccatggttccagaccgtgac

SEQ ID NO: 4 - DNA, 30 bp, Artificial Sequence
  Oligonucleotide PCR primer
aaatcatctagaattaccttatcctatctc

SEQ ID NO: 5 - DNA, 32 bp, Artificial Sequence
  Oligonucleotide PCR primer
gtcatggtctagaaaccatgcacgtaaaagag

SEQ ID NO: 6 - DNA, 36 bp, Artificial Sequence
  Oligonucleotide PCR primer
tacgtctagactatagatttatatttcttgttcgtt

(19) United States
(12) Patent Application Publication
(10) Pub. No.: US 2004/0013688 A1
(43) Pub. Date: Jan. 22, 2004

(54) Vaccines to induce mucosal immunity

(75) Inventors: Donald L. Wise, Belmont, MA (US); Debra J. Trantolo, Princeton, MA (US); David D. Hile, Medford, MA (US); Stephen A. Doherty, Newmarket, NH (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Cambridge Scientific, Inc.

(21) Appl. No.: 10/613,975

(22) Filed: Jul. 3, 2003

Related U.S. Application Data
(60) Provisional application No. 60/393,777, filed on Jul. 3, 2002.

(51) Int. Cl. A61K 39/02; A61K 39/002; A61K 9/16; A61K 9/50

(52) U.S. Cl. 424/190.1; 424/191.1; 424/490

(57) ABSTRACT

A bioadhesive mucosal delivery system is used in concert with systemic immunization to develop long-lasting immune responses correlative to protective immunity, especially for the prevention of infection with malaria, tularemia, anthrax, and H. pylori. First, the method provides controlled delivery of protective antigens, such as ODNs, to a mucosal site resulting in “priming” of mucosal receptors. Second, the method augments this mucosal prime with parenteral stimulation. In another embodiment, an intranasal vaccine is used in the treatment of tularemia and other bacterial and viral inhalation antigens. The use of CpG motifs in bacterial DNA allows for the activation of the innate immune response that is characterized by the production of immunostimulatory cytokines and polyreactive antibodies. The rapid response system limits the spread of the pathogen prior to specific immunity activation. The use of sustained mucosal exposure lowers the activation threshold of the innate immune system, allowing for a stronger and more rapid response to infection.

### CROSS-REFERENCE TO RELATED APPLICATIONS

This application claims priority to U.S. application Ser. No. 60/393,777, filed Jul. 3, 2002, entitled “Vaccines To Induce Mucosal Immunity” to Wise et al.

### BACKGROUND OF THE INVENTION

This is generally in the field of methods and compositions to induce persistent mucosal immunity to pathogens, especially those that may be used as bioterrorist weapons, and in particular vaccines utilizing a labile antigen such as a DNA plasmid.

There are many diseases, such as malaria, anthrax, and tularemia, which are primarily third world diseases, where there is limited access to preventative health care due to cost and few facilities and health care workers. These same diseases are also targeted by terrorist groups, because they are easily spread, there is limited immunity to the diseases, and large numbers can be quickly incapacitated or killed from exposure. Vaccines are the most efficient and cost-effective means for disease prevention. Twelve percent of the total costs for vaccination pays for the vaccine, while operational costs, such as personnel training, transportation, and maintenance of the cold chain, are responsible for the remainder of the costs. Clearly, such vaccines would be advantageous in the developing world and in the military where both would benefit from increased ease of mass immunization. However, for many diseases, few, if any, safe, effective and low cost vaccines are available.

Tularemia is a zoonotic disease caused by the bacterium Francisella tularensis. The disease predominates in the northern hemisphere. The expression of the disease is determined by the method of transmission. Oropharyngeal tularemia is observed following the ingestion of contaminated food or water. Oculoglander tularemia occurs when the bacteria contacts the conjunctiva of the eye. The most common expression is respiratory tularemia, which results from inhalation of contaminated dust. Respiratory tularemia, most prevalent in humans, is associated with select occupational groups and is often seen in local epidemic outbreaks. Despite the diverse methods of infection and infectious nature of the organism, the organism is not transmitted by infected individuals to others. Left untreated, the mortality rate is about 35 percent.

The highly infectious nature of tularemia, along with its stability and ease of production, make it a potential candidate for use as an effective biological warfare agent. Statistical tables by the World Health Organization (Anon, “Health aspects of chemical and biological weapons,” a report of WHO consultants, WHO, Geneva, 97-99 (1970)) indicate that an aerosol release of 50 kg of F. tularensis over a city of five million would result in incapacitating an estimated 250,000 persons, including 19,000 deaths, with illness persisting for weeks and periodic relapses for months. A 1997 report to the Centers for Disease Control and Prevention (CDC) estimated that the economic impact of a bioterrorist attack using F. tularensis would be $5.4 billion for every 100,000 persons exposed.

Although less virulent and fatal than anthrax or plague, F. tularensis has been considered as a biological weapon since at least the 1930's. Japan, the Soviet Union, and the United States are all known to have studied the organism for use as a biological weapon (Dennis, et. al., JAMA 285(21): 2763-2773 (2001)). Several outbreaks in Europe and the Soviet Union served to show the epidemic potential of this organism. Generally associated with rural areas, the largest inhalation tularemia outbreak occurred in a farming area of Sweden, with more than 600 reported cases. Although no deaths were reported, this indicates the virulence of the organism. The organism is known to survive for weeks in low temperature in a variety of environments, such as water, soil and hay. Humans can be infected by F. tularensis through the skin, gastrointestinal tract, lungs and mucous membranes. The major organs for attack are lymph nodes, lungs, kidneys and spleen. The organism spreads and multiplies in the lymph nodes, before dispersing to organs throughout the body.

Vaccines directed to F. tularensis require vaccination with live vaccine strain (LVS) to provide protection against the virulent form of the bacteria. Natural infection with F. tularensis also provides protection, while vaccination with non-viable or subfactions of non-viable cells are generally ineffective. Studies have shown a decrease in cases from 5.7 cases per 1000 person-years of risk to 0.27 cases per 1000 person-years of risk, when a non-viable vaccine was replaced with a live vaccine.

A vaccine directed toward thwarting F. tularensis would have an impact upon its potential to divert a bioterrorist threat, as well as bring about benign exposure to infection by it, or other pathogenic intracellular bacteria.

Each year approximately 300 to 500 million people are infected with malaria and each year 1.5 to 2.7 million people die from this disease. Since World War II, the struggle against malaria has gone through several stages. The first stage involved a massive effort aimed at eradicating the vector. The second stage was the development of antimalarial drugs based on quinine derivatives and alternatives. Due to introduced drug resistance, vaccination represents the best potential for control of the disease. The third stage of malaria control, then, recognizes the limitations of vector control and chemotherapy. In this regard, a current emphasis is on development of DNA-based vaccines against one or more of the developmental forms of the malaria parasite. Vaccines may prove beneficial to a wide range of populations. Proposed goals aim to prevent disease in foreign travelers and residents in low transmission areas such as India and reduce disease in high transmission areas such as sub-Saharan Africa. Even vaccines demonstrated to provoke only low levels of antibodies might be useful in priming the immune system. Subsequent natural infection would help reduce the disease in high-risk populations such as children and pregnant women of Africa.

The potential and applicability of malaria vaccines as a treatment method has led to the development of a number of candidates. Several additional candidate vaccines are expected in coming years upon sequencing of the P. falciparum genome (Gardner, et al., Science 282:1126-1132 (1998)). A successful malaria vaccine will eliminate the need for chemoprophylaxis in deployed troops and will prevent the degradation of fighting capabilities due to malaria infection. In addition, such a vaccine would protect civilian travelers and residents of malaria endemic areas.

Vaccine trials have progressed from mice (Doolan, et al., J. Exper Med 183:1739-1746 (1996)) to monkeys (Wang, et al., Infec Immun 66(9): 4193-4202 (1998)) and into humans (Stoute, et al., New Eng J Med 336:86-91 (1997); Wang, et al., Infec Immun 66(9): 4193-4202 (1998)). Malaria vaccines work by inducing the production of CD8⁺ T-cells that kill infected hepatocytes. Immunity stems from recognition of peptides present on the surface of infected hepatocytes by CD8⁺ T-cells that mediate infected cell elimination. Doolan et al., (J. Exp. Med 183: 1739-1746 (1996)) demonstrated partial protection ranging from 8 to 75 percent among various breeds of mice inoculated intramuscularly with DNA encoding for the Plasmodium yoelii circumsporozoite protein (PyCSP). Protection ranging from 80 to 90 percent was conferred onto mice by injection of a combination of plasmid vaccines, PyCSP and Plasmodium yoelli hepatocyte erythrocyte protein 17 (PyHEP17). The success of the combination was attributed to a circumvention of genetic restrictions that lessened protective immunity mediated by CD8⁺ T-cells. Clinical vaccines are likely to include several protein-inducing plasmids to overcome genetic restrictions and handle parasite polymorphism. The induction of antigen-specific antibodies required multiple immunizations. 8 of 12 animals expressed CD8⁺ T-cell responses to all of the delivered epitopes and three additional animals showed CD8⁺ T-cell responses to all but one. These results support the effectiveness of the multiple epitope immunization approach.

Based on the encouraging results in nonhuman primates, Hoffman, et al., (Immun Cell Biol 75: 376-381 (1997)) proposed a plan to clinically test a multigene malaria vaccine in humans. Twenty malaria naive volunteers were given three immunizations of the P. falciparum liver-stage DNA vaccine. The induction of CD8⁺ T-cells against the expressed protein was monitored by collection of peripheal blood mononuclear cells. Immune responses were detected in doses as small as 20 μg, but doses ranging from 500 to 2500 μg elicited responses to approximately 70 percent of all of the peptides studied. In general, the magnitude of the immune response was also reported to be significantly higher than observed in humans exposed to conventional irradiated sporozoites or natural infection alone. Le, et al., (Vaccine 18:1893-1901 (2000)) conducted safety studies and subjects observed mostly mild symptoms through one year following immunizations. However, the effectiveness of the vaccine was questioned, as there were no detectable antigen-specific antibodies present despite an induction of CD8⁺ T-cell response. Stoute, et al., (New Eng J Med 336:86-91 (1997)) conducted independent clinical trials of P. falciparum vaccines with mixed results. Human volunteers were vaccinated and then exposed to infection causing development of malaria in 100 percent of control subjects. Two vaccine formulations had little effect as the majority of volunteers contracted the disease, but a third formulation prevented malaria in seven of eight volunteers. Further studies were indicated to determine vaccine safety and reasons why the third formulation may have been more successful than others.

There have been attempts to improve vaccine efficacy. Sedegah, et al., (Proc. Nat. Acad. Sci., USA 95:7648-7653 (1998)) demonstrated increases in protection by priming with the malaria vaccine and boosting with recombinant vaccinia. Priming with PyCSP plasmid DNA and plasmid GM-CSF was demonstrated to confer protection to 100 percent of challenged mice dependent upon amount of recombinant vaccinia delivered during boosting.

Anthrax is an acute infectious disease caused by the spore-forming bacterium Bacillus anthracis. It occurs most frequently as an epizootic or enzootic disease of herbivores (e.g., cattle, goats, and sheep), which acquire spores from direct contact with contaminated soil. Humans usually become infected through contact with or ingestion of or inhalation of B. anthracis spores from infected animals or their products (e.g., goat hair). (Human-to-human transmission has not been documented.) The lethality of anthrax and the ease with which its spores can be disseminated has led military and counterterrorism planners to consider anthrax as one of the single greatest biological warfare threats. A WHO report (Anon, “Health aspects of chemical and biological weapons,” a report of WHO consultants, WHO, Geneva, 97-99 (1970)) estimated that three days after release of 50 kg of anthrax spores along a 2 km line upwind of a city of 500,000 population, 125,000 infections would occur, leading to 95,000 deaths within one to six days after exposure.

Anthrax is a well-known disease and was one of the first to be described in association with its causative organism, Bacillus anthracis (Koch, Mitt. Kaiserl. Gesundheits 1: 49-79 (1881)). Although the disease is well-characterized, only in recent years has the molecular basis of anthrax begun to be understood. The principal virulence factor of B. anthracis is a multicomponent toxin secreted by the organism that consists of three separate gene products, protective antigen (PA), lethal factor (LF), and edema factor (EF).

Typical DNA delivery methods rely on either intramuscular injection of soluble (active) DNA fragments (“plasmids”) or gene gun bombardment of particulate plasmids directly into recipient epithelial cells. Cellular responses vary tremendously depending on the delivery method with particulate bombardment often requiring several orders of magnitude less DNA to evoke immune responses (Pertmer, et al., Vaccine 13(15): 1427-1430 (1995)). However, to be of ultimate utility, the delivery system should be amenable to the targeting of appropriate immune responses in varying tissues appropriate to the pathogen exposure vis a vis mucosal vs. blood-borne pathogens. The strongest immune responses often develop at cellular levels commensurate with the route of exposure. Thus, there is a need for a DNA delivery system that optimizes the potency relative to the dose by supporting efficient transfection and expression via a variety of routes of administration so that an immune response appropriate to the exposure can be stimulated.

Mucous membranes are the primary routes of entry for a large number and wide variety of disease carrying agents including anthrax. Many human pathogens enter and replicate at the mucosal surface before causing systemic infection. It is particularly important to curtail infection at the mucosal surface before persistent infection of systemic sites or latency or chronic infection is initiated. Accumulated experimental evidence from animal models establishes the presence of a common mucosal immune system that may be stimulated by oral immunization. Oral immunization has been shown to result in the induction of secretory immunoglobulin and T cell responses at mucosal sites. In addition, stimulation of the mucosal immune system has been implicated in the development of systemic responses. Thus, oral immunization may be used for the induction of protective immunity against not only pathogens of the gastrointestinal (GI) tract, but also pathogens which infect at alternative mucosal sites. However, the induction of mucosal immunity following oral immunization has been shown to depend on a number of variables, including the dose and the nature of the antigenic component and the frequency of administration. One of the most crucial factors, then, in the success of oral immunization is the selection of the delivery system.

It is therefore an object of the present invention to provide a method and compositions to provide prolonged, improved protection against infectious pathogens, including P. falciparum, F. tularensis, H. pylori, and B. anthraci, especially using oral or intranasal routes of administration.

### BRIEF DESCRIPTION OF THE DRAWINGS

FIG. 1 is a graph of the release of VR2578 from PLGA particles in terms of time (days) versus percent impregnated pDNA released. Both empirical and theoretical measurements are represented in FIG. 1.

### SUMMARY OF THE INVENTION

A bioadhesive mucosal delivery system is used in concert with systemic immunization to develop long-lasting immune responses correlative to protective immunity, especially for the prevention of infection with malaria, tularemia, anthrax, and H. pylori. The method of vaccination serves two purposes. The first is the controlled delivery of protective antigens, such as oligodeoxynucleotides (ODNs), to a mucosal site resulting in “priming” of mucosal receptors. The second is to augment this mucosal prime with parenteral stimulation. In another embodiment, an intranasal vaccine is used in the treatment of tularemia and other bacterial and viral inhalation antigens. The use of CpG motifs in bacterial DNA allows for the activation of the innate immune response that is characterized by the production of immunostimulatory cytokines and polyreactive antibodies. The rapid response system activates to limit the spread of the pathogen prior to specific immunity activation. The use of sustained mucosal exposure has the added benefit of lowering the activation threshold of the innate immune system, allowing for a stronger and more rapid response to infection.

In the preferred embodiment, DNA plasmids are incorporated into a low molecular weight, biocompatible-hydrolytically labile (absorbable) poly(D,L-lactide-co-glycolide), PLGA-75:25 (Resomer 752). An open-celled polymeric foam, prepared by lyophilization (approximately 95% void volume), is impregnated with an aqueous solution of the plasmid. After a second lyophilization to remove the water, the matrix is extruded at ultrahigh pressures. High extrusion pressures trap the plasmid within the PLGA and minimize the early burst sometimes seen with matrix systems. The extrudate is then cryogenically ground to an average particle size of fifteen microns in diameter; ultrasonic sieving is then used to isolate particles less than five microns in diameter. A critical aspect of these formulations for inducing effective immunity in many diseases, such as tularemia, malaria and anthrax, is sustained and/or prolonged release over a period of weeks or months, to stimulate and maintain the immune response to the pathogens. The mucoadhesive coating enhances exposure to and uptake by the mucosal tissues, to further enhance and maintain the immune response.

### DETAILED DESCRIPTION OF THE INVENTION

I. Vaccine Compositions for Mucosal Immunity

Mucous membranes are the primary routes of entry for a large number and wide variety of disease-carrying agents, including tularemia. Many human pathogens enter and replicate at the mucosal surface before causing systemic infection. It is particularly important to curtail infection at the mucosal surface before persistent infection of systemic sites or latency or chronic infection is initiated.

Oral vaccines may stimulate mucosal immune systems to produce local immunoglobulin responses in addition to systemic responses. These vectors are delivered to the mucosal surface, the site where the infection actually occurs.

The prevailing view in the field of mucosal immunology has been that induction of mucosal immune responses requires that antigens be introduced to mucosal-associated lymphoid tissue (MALT). The main mucosal inductive sites are the gut-associated lymphoid tissue (GALT) and the nasal-associated lymphoid tissue (NALT). The indicator of a mucosal immune response is the local production and secretion of the IgA isotype of the immunoglobulin family.

To date, most studies of MALT responses have focused on the GALT where the follicle-like structures of the Peyer's patches covered with M cells have been shown to be responsible for sampling of the antigen (deHaan, et al., Immun. Cell Biol. 76: 270-279 (1998)). This sampling results in transcytosis of the antigen to antigen-presenting cells located in the dome area of the follicles. This dome area contains B and CD4⁺ T cells which, when stimulated, migrate to the lymph nodes where they proliferate prior to entering the circulation and traveling to mucosal effector sites (Neutra, et al., Annu. Rev. Immunol. 14: 275-300 (1996)). The interconnected mucosal system thus stimulated is referred to as the common mucosal immune system (CMIS). It is during this effector phase of the mucosal immune response that IgA is made. It is assumed that the NALT operates in a similar fashion, i.e., stimulation of the CMIS, but less is known of the anatomy and the function of the NALT (Kuper, et al., Immunol. Today 13: 219-224 (1992); Wu, et al., Immunol. Res. 16: 187-201 (1997)).

Although enormous amounts of IgA are made when the immune system is stimulated, it has been difficult to develop this immune response using soluble or non-replicating antigens. Local administration usually does not produce a response or requires large amounts of antigen to produce a response (McGhee, et al., Vaccine 10: 75-88 (1992)). This effect is further complicated if there has been prior exposure to the antigen, which often leaves the receptor in a state of immunological non-responsiveness or tolerance (Weiner, Proc. Natl. Acad. Sci. USA 91: 10762-10765 (1994)).

Clearly, the delivery of antigen is key to developing the immune response. Delivery must thus address not only the mode of presentation, but also the rate and dose of antigen. Under-stimulation may fail to prime the system and over-stimulation may result in tolerance. Although previous studies on mucosal vaccine development have focused on the sole manipulation of mucosal delivery, vis à vis exploring various mucosal sites or toxin adjuvants, there is emerging evidence that a protective mucosal response may, in fact, be achieved by combining mucosal administration of antigen with parenteral administration.

In some cases, natural mucosal priming appears to be a prerequisite for effective parenteral vaccination and may be the reason for disparity in the immunoresponsiveness of clinical trial groups. In a study of parenteral vaccination against influenza, for example, naturally primed adults and primed children (determined on the basis of prevaccination serum antibodies) had significantly higher IgG and IgA responses than unprimed children (el-Madhun, et al., J. Infect. Dis 178(4): 933-999 (1998)). Indeed, parenteral immunization now appears to be a viable route for vaccination against H. pylori in those populations with prior exposure to H. pylori (equivalent to a “natural mucosal prime”) (Guy, et al., Vaccine 17(9-10): 1130-1135 (1999), Vaccine 16(8): 850-866 (1998a)).

In addition, mucosal systems can be synthetically primed as shown by Lee, et al., (Vaccine 17(23-24): 3072-3082 (1999)) where naïve primates were effectively immunized against H. pylori using a vaccination protocol that combines a mucosal prime with parenteral boosts. This technique is showing promise for other indications as well, e.g., flu vaccines (Guy, et al., Clin. Diagn. Lab. Immunol. 5(5): 732-736 (1998b), Vaccine 16(8): 850-866 (1998a)).

The method and delivery systems for the delivery of DNA vaccine encoding antigens to the mucosal associated lymphoid tissue (MALT) have been developed which overcome these limitations. Lymphoid follicles with microfold (M) cells are particularly numerous in the distal colonic and rectal mucosa of humans. However, for any mucosal site, uptake of antigen is a critical step in the generation of mucosal immunity, based on the stimulation of antibody secreting cells and helper T cell subsets in the lymphoid follicles of the gut and other mucosal tissues. For efficient induction of mucosal immunity it is necessary to present antigens in particulate form to specialized M-cells, which are present at highest density in follicular domes of the MALT. This is achieved by incorporation of the antigens into particulates using an extremely gentle method which does not denature the antigens, and yet presents large quantities of antigen to the mucosal tissue.

In the preferred embodiment, the antigen is a nucleic acid molecule encoding a protein antigen that induces immunity. In the most preferred embodiment, the antigen is a DNA plasmid molecule. Plasmid DNA vaccines incorporating the DNA into absorbable polymers are more likely to be effective than injections of the naked plasmid. This effect arises from the slow release from the system. In addition to this immunological advantage, there are practical benefits to injectable controlled vaccines. These include easier administration and ‘unlimited’ frequency of boosting (if necessary) because these vaccines reduce the need for trained personnel to deliver the vaccines. The cost of vaccines to the health care industry, at large, and to the military and developing country markets, specifically, is an important issue. The development of less expensive vaccines would have a significant impact upon the extent of vaccine coverage throughout these markets.

A. Antigens

Suitable antigens are known and available from commercial, government, and scientific sources. In the preferred embodiment, the antigens are DNA plasmids encoding all or part of a viral or bacterial protein. Specific examples are described below. Antigen is preferably administered with an adjuvent such as ODNs, alum, or other adjuvents which are approved for administration to humans. Synthetic oligodeoxynucleotides containing CpG motifs has been shown to simulate protection against lethal infection (Elkins, et al., J. Immunol. 162(4): 2291-2298 (1999)). The synthetic ODNs induce the lymphocytes and macrophages to produce polyreactive antibodies and/or cytokines, including the gamma interferon (IPN-γ). (Klinman, et al., Infect Immun 67: 5685-5663 (1999)).

P. falciparum

Malaria vaccines work by inducing the production of CD8⁺ T-cells that kill infected hepatocytes. Immunity stems from recognition of peptides present on the surface of infected hepatocytes by CD8⁺ T-cells that mediate infected cell elimination. Antigens which have been effective in inducing immunity include DNA coding for the Plasmodium yoelii circumsporozoite protein (PyCSP). Protection ranging from 80 to 90 percent was conferred onto mice by injection of a combination of plasmid vaccines, PyCSP and Plasmodium yoelli hepatocyte erythrocyte protein 17 (PyHEP17).

Plasmids available from NMRC include: VR2516 (native PyCSP in 1020), VR2515 (native PyHEP17 in 2020), VR2578 (synthetic PyCSP in 1020), VR2579 (synthetic PyHEP17 in 1020), VR2533 (native PyMSP1 in 1020), and VR1020 (control plasmid).

Stoute, et al., (New Eng. J. Med 336:86-91 (1997)) evaluated three formulations of a recombinant circumsporozoite protein vaccine, RTS,S (SmithKline Beecham Biologicals, Belgium). Vaccine RTS,S consists of two polypeptides that simultaneously form composite particulate structures on their simultaneous synthesis in yeast (Saccharomyces cerevisae). RTS is a single polypeptide chain derived from P. falciparum (3D7) that is fused to HBsAg (and serotype). S is a polypeptide that corresponds to HBs/Ag. Formulations were prepared in several vehicles. Vaccine 1 was contained in alum plus monophosphoryl lipid A, vaccine 2 in an oil in water emulsion, and vaccine 3 in the same emulsion, but containing two immune stimulants, one of which was the monophosphoryl lipid A. The vaccines were administered to healthy volunteers at 0, 4, and 28 weeks. IgG antibody titers peaked at about day 44 of the study and remained fairly constant thereafter.

Sedegah, et al., (J. Immun. 164: 5905-5912 (2000)) showed that protective immunization in mice by injection of naked plasmid DNA expressing P. yoelii circumsporozoite protein (PyCSP) could be improved either by coadministration of a plasmid expressing murine GM-CSF or by boosting with recombinant poxvirus expressing the PyCSP. Boosters were given at 3, 6, 9, or 12 weeks after priming with DNA.

Accordingly, in the preferred embodiment, the antigens are plasmids encoding multiple P. yoelii proteins, administered in a formulation providing release over a period of at least 3, 6, 9 or 12 weeks, most preferably after release an initial priming dose or administered with a priming dose.

F. tularensis

Vaccination with live vaccine strain (LVS) or natural exposure to infection of F. tularensis provides protection against tularemia. The use of non-viable cells or subfactions of non-viable F. tularensis does not provide protection against a virulent form of the bacteria. Synthetic ODNs that express CpG motifs and mimic the immunostimulatory properties of bacterial DNA are preferred as the antigen for F. tularensis. These can be obtained from Dennis M. Klinman, Ph.D., CBER/FDA. The synthesized ODN has the sequence GCTAGACGTTAGCGT (SEQ ID NO: 1) and TCAACGTTGA (SEQ ID NO: 2). All ODN can be tested for endotoxin content by chromogenic Limulus ameobocyte lysate assay and for protein contamination by the bicinchoninic acid protein assay kit (Pierce Chemicals, Rockford, Ill.) (Klinman, et al., Infect. Immun. 67: 5685-5663 (1999)).

Repeated administration of synthetic ODNs expressing CpG motifs has been shown to provide protection against F. tularensis for up to two weeks (Klinman, et al., Infect. Immun. 67: 5685-5663 (1999)). By repeatedly administering CpG ODN for two to four times per month, protection can be maintained indefinitely (Klinman, et al., Infect. Immun. 67: 5685-5663 (1999)). The cellular basis of DNA protection has been studied in mice with genetic defects. There were no survivors of mice that were lymphocyte deficient after treatment with an ODN, followed by 100-1000 LD₅₀ challenge. Those treated with LVS DNA had a survival rate of 82 percent. This indicates that B cells are crucial to DNA mediated protection (Elkins, et al., J. Immun 162(4): 2291-2298 (1999)). Numerous studies have shown that CpG motifs initiate Ag-specific immunity. Additional studies have shown that DNA treatment was able to induce pathogen specific immunity in a manner similar to immunization with subleathal bacterial infection (Elkins, et al., Infect. Immun 60(11): 4571-4577 (1992), Elkins, et al., Microb. Pathog. 13(5): 417-421 (1992), Yee, et al., J. Immunol. 157: 5042-5048 (1996)). Extension of this protection was possible through repeated ODN administration. Protection was maintained with weekly treatments for a period of four months, however the protection was lost one month after treatment was discontinued.

It is believed that the use of CpG ODN enables the introduction of protection for a variety of pathogens and that with repeated administration long-term protection can be achieved (Elkins, et al., J. Immun. 162(4): 2291-2298 (1999), Krieg, et al., J. Immunol. 161: 2428-2434 (1998)). The need for repeated dosing makes an extended controlled release system a highly desirable and advantageous complement for this treatment method. Therefore, in a preferred embodiment, the antigen is incorporated into a particulate formulation providing sustain, prolonged release, for at least two weeks, one more, or more preferably longer.

Anthrax

The principal virulence factor of B. anthracis is a multicomponent toxin secreted by the organism that consists of three separate gene products, protective antigen (PA), lethal factor (LF), and edema factor (EF). The genes encoding these toxin components (pag, lef, and cya, respectively) are located on a 184-kb plasmid designated pXO1, carried by all strains of B. anthracis (Mikesell, et al., Infect. Immun. 39: 371-376 (1983)). PA (735 amino acids [aa]; Mᵣ, 82,684) is a single-chain protein which binds to an as yet unidentified receptor on the cell surface and subsequently undergoes furin-mediated cleavage to yield a 63-kDA receptor-bound product (Gordon, et al., Infect. Immun 63: 82-87 (1995); Klimpel, et al., Proc. Natl. Acad. Sci., USA 89: 10277-10281 (1992); and Leppla, et al., Bacterial protein toxins (Fehrenbach, et al., eds) pp. 111-112, Gustav Fischer: NY, 1988). The 63-kDa PA fragment forms a heptameric complex on the cell surface which is capable of interacting with either the 90-kDA LF protein or the 89-kDA EF-protein, which is subsequently internalized (Milne, et al., J. Biol. Chem 269: 20607-20612 (1994)). LF (776 aa; Mᵣ 90,237) is a zinc metalloprotease that cleaves several isoforms of mitogen-activated protein kinase kinase (Mck1, Mck2, and MKK3), thereby disrupting signal transduction events within the cell and eventually leading to cell death (Duesbery, et al., Science 280: 734-737 (1998)). The EF protein is a calmodulin-dependent adenylate cyclase that causes deregulation of cellular physiology, leading to clinical manifestations that include edema (Leppla, Proc. Natl. Acad. Sci. USA 79: 3162-3166 (1982)). The LF protein combines with PA to form what is referred to as lethal toxin (Letx), which is considered to be the primary factor responsible for the lethal outcome of anthrax infection.

Protection against anthrax infection is associated with a humoral immune response directed against PA. Some evidence suggest that EF and LF may also contribute to specific immunity (Ivins, et al., Eur. J. Epidemiol. 4: 12-19 (1988); Little, et al., Infect. Immun. 52: 509-512 (1986)), although these components have not been formulated into a subunit vaccine. One can obtain a protective response to a lethal toxin (Letx) challenge by immunization with a plasmid encoding the 63-kDa protease-cleaved fragment (PA₆₃) of PA (Gu, et al., Vaccine 17: 340-344 (1999); Price, et al., Infect. Immun. 69(7):4509-4515 (2001)). Combined immunization with genes encoding PA and LF can also provide additional protection against the effects of Letx.

The minimum PA and LF structures which could form a functional binding complex while eliminating the metalloprotease function of LF can be carried out using the gene fragment encoding PA₆₃, which is capable of binding to the PA receptor and to LF, and the gene fragment encoding LF4 (aa 1 to 254), which contains the N-terminal and one-third of the LF antigen, but lacks the domain associated with the LF metalloprotease function yet retains the ability to bind to (PA₆₃) (Arora, et al., J. Bio. Chem 268: 3334-3341 (1993); Gupta, et al., Biochem. Biophys. Res. Commun. 280: 158-163 (2001)). The eucaryotic expression plasmid pCI (Promega, Inc., Madison, Wis.) is used to study for the expression of truncated versions of the PA and LF proteins. The gene fragment coding aa 175 to 764 of the PA protein is PCR amplified using the plus-strand primer 5′-ACA AGT CTC GAG ACC ATG GTT CCA GAC CGT GAC-3′ (SEQ ID NO: 3) and the minus-strand primer 3′-CTC TAT CCT ATT CCA TTA AGA TCT ACT AAA-5′ (SEQ ID NO: 4), with pYS2 as a template. Included in the primer sequences are Xho1 and Xba1 restriction sites (undefined), respectively. The PA gene fragment corresponds to the biologically active, protease-cleaved PA₆₃ fragment of the full length 83-kDA protein (Gordon, et al., Infect. Immun. 63: 82-87 (1995)). The PCR product is digested with Xho1 and Xba1 and ligated into the pC1 vector, which has been cut with the same two restriction enzymes. The plasmid construction pCLF4 encodes aa 10 to 254 of LF, which constitutes the PA binding domain. This plasmid is constructed from a PCR-amplified fragment using the primers 5′-GT CAT GGT CTA GAA ACC ATG CAC GTA AAA GAG-3′ (SEQ ID NO: 5) and 3′-TTG CTT GTT CTT TAT ATT TAG ATA TCA GAT CTG CAT-5′ (SEQ ID NO: 6), which incorporates Xba1 cleavage sites (underlined). The Xba1-digested PCR and pCI plasmid fragments are ligated to form the pCLF4 plasmid. Neither of the resulting plasmid constructs, pCPA and pCLF4, contain a signal sequence for secretion of the expressed gene product. All plasmids are purified from Escherichia coli DH5α using Endo-free plasmid preparation kits (Qiagen) and are dissolved before use in phosphate-buffered saline (0.15 M NaC1, 0.01 M Na phosphate, pH 7.3).

The ability of genetic vaccination to protect against a lethal challenge of anthrax toxin was evaluated. BALB/c mice were immunized via gene gun inoculation with eucaryotic expression vector plasmids encoding either a fragment of the protective antigen (PA) or a fragment of lethal factor (LF). Plasmid pCLF4 contains the N-terminal region (amino acids [aa] 10 to 254) of Bacillus anthracis LF cloned into the pCI expression plasmid. Plasmid pCPA contains a biologically active portion (aa 175 to 764) of B. anthracis PA cloned into the pCI expression vector. One-micrometer-diameter gold particles were coated with plasmid pCLF4 or pCPA or a 1:1 mixture of both and injected into mice via a gene gun (1 μg of plasmid DNA/injection) with each of three immunizations at 2-week intervals. Sera were collected and analyzed for antibody titer as well as antibody isotype. Significantly, titers of antibody to both PA and LF from mice immunized with the combination of pCPA and pCLF4 were four to five times greater than titers from mice immunized with either gene alone. Two weeks following the third and final plasmid DNA boost, all mice were challenged with five LD₅₀ doses of lethal toxin (PA plus LF) injected intravenously into the tail vein. All mice immunized with pCLF4, pCPA, or the combination of both survived the challenge, whereas all unimmunized mice did not survive. These results demonstrate that DNA-based immunization alone can provide protection against a lethal toxin challenge and that DNA immunization against the LF antigen alone provides complete protection.

In the preferred embodiment, the oral dose forms comprise the primary carrier (bioadhesive/plasmid/PLGA microparticles) and the secondary carrier designed to bring the active particles to the colon. The plasmid (PA and LF) is incorporated into a primary PLGA carrier designed to release the PA at different rates for stimulation of three distinct antibody peaks (simulating dosing).

B. Controlled, Sustained and/or Prolonged Release Carrier Systems for Mucosal Delivery

The effectiveness and longevity of vaccines, especially vaccines incorporating nucleic acid encoding antigen, such as plasmid DNA, may be improved by incorporation of pDNA within polymeric delivery vehicles. Administration of naked pDNA leaves the vaccine vulnerable to attack from degradation enzymes that can reduce half-lives to minutes or hours (Kawabata, et al., Pharm. Res. 12(6): 825-830 (1995), Luo, et al., Nature Biotech 18: 33-37 (2000)). Chemical modification of DNA has previously been utilized to protect the vaccine from nucleases and increase vaccine longevity (Benns, et al., J. Drug Target. 8(1): 1-12 (2000), Luo, et al., Nature Biotech. 18: 33-37 (2000)). Modified vaccines have been complexed with cationic and anionic liposomes, polysaccharides, poly(ethylene glycol), and poly(L-lysine) among others. A drawback to chemical modification has been increases in systemic toxicity resulting from exposure to the complexed chemicals (Luo, et al., Nature Biotech. 18: 33-37 (2000)).

A second alternative involves encapsulation of the plasmid within a polymeric carrier. Biodegradable homo- and copolymers of lactide and glycolide (the “PLGAs”) provide protection for the plasmid, while enabling a sustained and controlled release of the plasmid. Anchordoquy, et al., (J. Pharm. Science. 89(3): 289-296 (2000)) reviewed the stability of plasmid-based therapies and suggested that polymeric carrier vehicles such as copolymers of lactide and glycolide (PLGA) may have potential to isolate and entrap DNA. Isolation of the plasmid may prove to be beneficial in reducing negative interactions such as aggregation that leads to loss of biological activity in typical liquid formulations.

The use of homo- and copolymers of lactide and glycolide for biomedical applications is well-established and is based on the biocompatibility of these materials and their degradation products, lactic and glycolic acids (Visscher, et al., J. Biomed. Mat. Res. 19: 349-365 (1985)). Rates of degradation and release of incorporated active agents are dependent both on the molecular weight of the polymer and on the lactide-to-glycolide ratio. Control of plasmid release may improve vaccine efficacy because prolonged availability may enable sustained gene expression (Labhasetwar, et al., J. Pharm. Science. 97(11): 1347-1350 (1998)).

Recent researchers have studied the encapsulation of plasmid-based therapeutics within polymer-based vehicles. Tinsley-Bown, et al., (J. Con. Rel. 66: 229-241 (2000)) demonstrated the release of a firefly luciferase-derived plasmid from microcapsules of a PLGA. In vitro studies found that the release rate of the plasmid into solution was dependent upon polymer molecular weight. Perez, et al., (J. Con. Rel. 75(1 and II): 211-224 (2001)) encapsulated plasmid DNA into nanoparticles of poly(lactic acid) and poly(ethylene glycol) copolymers. In this study, plasmid loadings of 10-12 μg per mg of polymer resulted in a large initial burst of plasmid from the matrix followed by a slower release for 28 days.

Traditional emulsion techniques for PLGA vaccines use blenders to generate the emulsions. However, the energy of this process results in some degradation of the DNA. As a consequence, a large portion of the supercoiled material is degraded to the open circle or linear form. The damage is a consequence of the shear forces acting on the liquid components of the emulsion.

Whereas polymeric carriers provide advantages over naked pDNA injections, loss of vaccine effectiveness in terms of physical mass loss and structural rearrangement of pDNA has been observed for encapsulation within polymeric delivery vehicles.

a. Methods for Encapsulation

Encapsulation efficiency of pDNA within PLGA matrices has varied with technique. Various procedures modified from the traditional double emulsion/solvent evaporation technique have yielded encapsulation efficiencies in the range of 20-50 percent (Tinsley-Brown, et al., J. Con. Rel. 66: 229-241 (2000) and 30-35 percent (Capan, et al., Pharm. Res. 16(4): 509-513 (1999)). However, Cohen, et al., (Gene Ther. 7: 1896-1905 (2000)) reported a higher efficiency, 70 percent, for encapsulation of pDNA within nanoparticles of PLGA than otherwise found. In addition to mass loss during the encapsulation procedure, rearrangements of pDNA structure have also been reported. A significant decrease in the percentage of supercoiled pDNA in favor of open circle pDNA has been reported. Tinsley-Brown, et al., (J. Con. Rel. 66: 229-241 (2000)) reported that 30-40 percent of pDNA was recovered in the supercoiled form with losses being attributed to the open circle conformation. Capan, et al., (Pharm. Res. 16(4): 509-513 (1999)) observed an increased loss of supercoiling, 16 percent, for uncomplexed pDNA. However, through forming of pDNA-poly(L-lysine) complexes, the percentage of pDNA remaining in the supercoiled structure increased to 75-85 percent.

The preferred carrier system is made as described in U.S. Pat. Nos. 5,456,917 and 5,429,822 to Wise, et al. The technology relies on solid-state matrix formulation methods, rather than encapsulation methods, to produce a biocompatible, degradable micron-sized particulate with adjuvancy and bioadhesion. Particle size reduction is accomplished by low temperature grinding (−40° to −50° C.) of solid particles in which shear forces on liquid droplets do not occur. The impact on solid particles transfers energy to the particle that dissipates on fracture and results in only a transient temperature rise. Thus, denaturation, or other destructive processes are limited. This system preserves protein antigenicity during formulation (biologicals are dispersed within the polymer matrix using aqueous or other stabilizing media) and more easily adapted to incorporation of bioadhesives (where enhanced adhesion may augment the immune response).

b. Effects of PLGA Vehicle on pDNA

The effectiveness of pDNA vaccines delivered in a PLGA vehicle has been demonstrated in vivo. Cohen, et al., (Gene Ther. 7: 1896-1905 (2000)) showed that a sustained release of pDNA from PLGA microparticles increased expression of alkaline phosphatase versus an injection of naked pDNA beyond 7 days. However, injections of polymer-encapsulated pDNA resulted in less expression versus naked pDNA for a period of 72 hours post-injection. This observation was attributed to the reduced availability of encapsulated pDNA with respect to the naked pDNA solution or diminished effectiveness of the vaccine due to rearrangements of pDNA structure. Yet, the polymeric delivery vehicle enables sustained release of pDNA vaccine. Lunsford, et al., (J. Drug. Target. 8(1): 39-50 (2000)) demonstrated persistence of pDNA within specific tissues in mice for a period of 120 days following injection for intramuscularly or subcutaneous injections. Tissues exposed to injections of naked pDNA were observed to be absent of pDNA beyond 15 days post-injection. Vaccine effectiveness may also be benefited by the potential of the polymeric particles to mediate transfection of macrophages during phagocytosis (Cohen, et al., Gene Ther. 7: 1896-1905 (2000)).

c. Characteristics of PLGA Particles

Plasmid DNA can be encapsulated into poly(lactide-co-glycolide) (PLGA, Resomer 752, 75:25-PLGA) microparticles. The polymer particle diameter is less than 50 μm as estimated by light spectroscopy. The concentration of pDNA in the polymer particles was determined following NaOH (aq.) digestion and spectrophotometric analysis of the aqueous phase to be approximately 10 μg per mg polymer with an encapsulation efficiency of approximately 100%.

Thomason, et al. (J. Cont. Rel. 41: 131-145 (1996)) reported delivery of P30B2 for 49 days with a second burst maximum at 28 days from 50:50-PLGA microspheres. Microspheres prepared from the slower degrading 75:25-PLGA released for 56 days with a second burst maximum to 42 days. Tinsley-Bown, et al., (J. Con. Rel. 66: 229-241 (2000)) reported very similar release patterns from 50:50-PLGA microparticles with 100% release at 42 days. The release patterns were similar in that early release was followed by virtually no release until the second burst.

d. Polymers

Biodegradable polymers are preferred for the delivery of vaccines for both parenteral delivery and mucosal delivery (Davis, Res. Immunol. 149: 49-52 (1998)). A number of biodegradable polymers are known, including natural polymers such as proteins like gelatin and albumin and polysaccharides like chitosan, dextrans, and celluloses. There are a large number of synthetic polymers that can be used, including polyhydroxy acids (such as polylactic acid, polyglycolic acid and copolymers thereof), polyanhydrides, polyorthoesters, and polyhydroxyalkanoates such as polybutyric acid. Although PLA, PGA and copolymers thereof are examples of biodegradable polymers, one of ordinary skill in the art will appreciate that other polymers, such as polydioxanone, poly(ε-caprolactone), polyanhydrides, poly(ortho esters), poly(ether-esters), polyamides, polylactones, poly(propylene fumarates), and combinations thereof, may be similarly used. The polymers can also include excipients such as surfactants, buffers, bioadhesives, plasticizers, salts, pore forming agents, and other additives commonly used in the manufacture of biocompatible polymeric drug delivery devices.

Polylactic-co-glycolic acid (PLGA) is a preferred polymer. In addition to the advantages owing to the particulate (and sometimes adjuvant) nature of PLGA dose forms, there is sustained release of the active agent. Incorporation of the active agent into the polymer commonly utilizes encapsulation techniques (e.g., Eldridge, et al., Infec. Imm. 59: 2978-2986 (1991); O'Hagan, et al., Vaccine 9: 768 (1991), O'Hagan, et al., Vaccine 11(9): 965-969 (1993); Singh, et al., Pharm. Res. 8: 958-961 (1991); Gilley, et al., Proc. Int. Symp. Cont. Rel. Bioact. Mater. 19: 110 (1992); Alonso, et al., Pharm. Res. 10: 945 (1993); Partidos, et al., J. Imm. Meth. 195: 135-138 (1996)). The quantity of material that can be encapsulated using conventional emulsion-based microencapsulation techniques is commonly quite small (1 to 10 percent).

e. Bioadhesives

In another embodiment, a bioadhesive is added to the vaccine carrier. Two types of PLGA matrices were initially prepared for this work: one combining antigen with PLGA exclusively, the other combining antigen with PLGA and gelatin. The former exploited considerations of particulate size and antigen controlled release; the latter addressed the additional potential of matrix bioadhesion. The optimization of particle uptake utilized both in vitro bioadhesion tests and in vivo ligated intestinal loop protocols. PLGA matrices were screened at various gelatin loadings using the model polypeptide, poly(L-lysine) labeled with a fluorescent marker, fluorescein isothiocyanate (PLL-FITC), at a loading of 1 percent in PLGA (w/w) and 0, 1, 3, or 10 percent (w/w) type A gelatin.

Adhesion data were subjected to a single factor analysis of variance (ANOVA), the single parameter being the gelatin content. Each data set was compared individually with the control (0 percent gelatin) and an f-value computed. Although the mean adhesion force was, in all cases, greater than that of the control, the null hypothesis (no significant difference between means) could not be rejected for the 3 percent and 10 percent gelatin levels. Although increased adhesion was observed at all gelatin levels, the formulation containing 1 percent gave statistically significant better adhesion at the 95 percent confidence limit. At 1 percent loading an increase of adhesive force of 58 percent was observed.

M-cell adherence of the various sample preparations was assessed in vivo using murine ligated intestinal loops (Ermack, et al., Cell Tiss. Res. 279: 433436 (1995)). Of the samples tested, particles consisting of PLGA/FITC+1 percent gelatin had the greatest frequency of binding to the M-cell-containing dome region. Particles with no bioadhesive only were rarely observed adhering to the dome region.

The number of particles bound to the dome region of the Peyer's patch was assessed. There was either binding of 5-10 particles/dome or binding of 0 particles/dome. In the presence of the bioadhesive, gelatin, the greatest number of adherent particles was detected. Of the samples tested, particles consisting of PLGA/PLL-FITC and 1 percent gelatin had the greatest frequency of binding to the M-cell-containing dome region. Particles consisting of PLGA/PLL-FITC only were rarely observed adhering to the dome region. Results indicate that particles including gelatin bound more effectively than those without gelatin.

Samples were viewed by fluorescent microscopy (FITC, TRITC channels) to distinguish fluorescent particles from the autofluorescent granulocyte cell populations generally located in the subepithelial region of the Peyer's patch dome. Particles fluoresced on the FITC channel, but not on the TRITC channel. In contrast, autofluorescent granulocytes were visible on either channel. M-cell adherence was detected only with PLGA/PLL-FITC and 1 percent gelatin.

The plasmid/PLGA dose form (modified with a bioadhesive and also containing an adjuvant) may be contained within a secondary carrier comprised of soluble gelatin capsules coated with Eudragit S that is insoluble below pH 7. The Eudragits (Rohm Tech, Malden, Mass.) are copolymers of acrylic acids and acrylic acid esters and are used in pharmaceutical preparations as enteric coatings for tablets and crystals.

Presentation of plasmids to the immune system following endocytosis depends on the rate of release of the plasmids from the excipient, which in turn depends on the properties enumerated above. Delivery depends on the rate of release and of hydrolytic degradation of the excipient.

pDNA/PLGA Matrix: In Vitro Release of DNA

In work to test an HIV vaccine, transfection and expression of immunogens with an associated immune response following intramuscular administration of a DNA/PLGA particulate using a well characterized DNA plasmid vector. Two DNA constructs were used: pJW/β-gal and pJW/env. Both use the plasmid backbone, pJW40632, which contains a cytomegalovirus promoter to allow gene expression in mammalian cells. A β-galactosidase (β-gal)-encoding gene, derived from the pcDNA3 plasmid or the full-length Rauscher Leukemia Virus (RLV) env gene, was inserted into the splice site of the pJW40632 plasmid.

The structural integrity of the plasmid DNA in each of the PLGA/DNA biopolymer preparations was tested by restriction enzyme analysis of released material. Four mg of a PLGA/pJW-β-gal preparation was incubated in normal saline plus EDTA (1 mM) at 25° C. The estimated DNA loading in this preparation was 5% (w/w). At 24 hours, the saline solution was removed, the PLGA/DNA biopolymer washed 3 times and then reincubated in fresh saline/EDTA. For each sample, an aliquot sample of the saline solution was treated with ethanol to precipitate any DNA released into the solution from the biopolymer/DNA complex. Ethanol precipitated material was resuspended in Tris/EDTA, digested with the restriction endonuclease, Hind III, and analyzed by ethidium bromide agarose gel electrophoresis.

Release of DNA

The majority of the PLGA-incorporated DNA was released in the first 24 hours, followed by the continued release of DNA over the next 48 hours and then much less DNA was released over the following 6 weeks. Importantly, intact DNA, identified by having a molecular weight identical to that of unincorporated starting DNA following endonuclease digestion at a single site on the plasmid, was recovered throughout the 6 week incubation period.

Functional Integrity of Released DNA

Following gel analysis for DNA release, aliquot samples of ethanol precipitated DNA from each DNA/PLGA preparation were tested for biological activity. DNA released from complexes was used for transient transfection of Cos 7 cells using DEAE-dextran. For pJW/env/PLGA, transfected cells were lysed 3 days later, lysates were run on polyacrylamide gels, proteins were transferred to nitrocellulose membranes, and the presence of the Env glycoprotein, coded for by the plasmid DNA and produced in transfected cells, was identified by immunoblotting using anti-Rauscher Leukemia Virus (RLV) antiserum. These results were compared with the results obtained from unincorporated plasmid DNA. Both the incorporated and unincorporated pDNA yielded similar levels of Env glycoprotein. For pJW/β-Gal/PLGA complexes and unincorporated pJW/β-gal, transfected cells were directly stained for β-galactosidase activity using X-gal colorimetric substrate to confirm expression. These results demonstrated that both the incorporated and unincorporated pJW/β-gal were functionally expressed.

The overall findings demonstrate that DNA expression plasmids can be successfully incorporated into PLGA biopolymers under aseptic conditions and released without loss of the biochemical or functional integrity of the DNA.

In Vivo Results Using DNA/PLGA Complexes

Inoculation of DNA/PLGA complexes into mice generated real antibody responses to proteins encoded by the DNA. Comparison to antibody responses obtained from immunization with soluble plasmids suggests that generation of antibodies is dose (and release) dependent. Animals dosed at 10 μg soluble DNA showed positive responses in all mice; animals dosed at 50 μg soluble DNA, however, showed inconsistent responses. Findings of measurable antigen-specific humoral responses in mice inoculated with DNA/PLGA biopolymers demonstrate that using PLGA biopolymers for making a one-shot prime/boost vaccine is workable. Wolff, et al., (Science 247(1): 1465-1468 (1990)) demonstrated that proteins coded for by injected DNA are expressed in muscle fibers after inoculation directly into the tissue. In some reports, DNA amounts as low as 5-10 μg/injection resulted in reporter gene expression.

Bioadhesive matrices can be prepared for immediate, intermediate, and prolonged delivery of the antigen, such as ODN, from gelatin/PLGA mucosal carriers. In a preferred embodiment, ODN are incorporated into polymeric foam, which also contains a mucoadhesive, such as gelatin, for bioadhesion as previously described by Trantolo, et al, (Proceedings of the Fifth World Congress, Chemical Engineering (1996)); Hsu, et al., (J. Biomed. Mat. Res. 35: 107-116 (1997)); Smith, et al., (Oral. Microbiol. Immun. 15: 124-130 (2000)). A low molecular weight, 75:25 PLGA polymer is used. Polymers with this composition, accepted for medical use and available from BI Chemicals, Inc. (Wallingford, Conn.) are marketed as Resomer RG 752. A polymer foam is first prepared by lyophilization of a solution of approximately 50 mg/ml in glacial acetic acid. This yields an open celled foam of approximately 60 mg/cm³ density. Gelatin and ODN are incorporated by impregnating the foam with an aqueous solution by a series of evacuations and repressurizations. The gelatin/ODN content is related to the solution concentration by the following relationship:

F=[1+dₚd_{f}/C(dₚ−d_{f})]⁻¹ Eq. 1

where

F=weight fraction of gelatin

dₚ=material density of nonporous polymer (g/cm³)

d_{f}=density of polymer foam (g/cm³),

C=concentration of gelatin solution (g/cm³).

To prevent migration of the ODN and gelatin to the particle surface, removal of the solvent water is accomplished by freeze-drying. Following this step the matrix is compressed at a pressure of 38,200 psi and at a temperature just above the glass transition temperature of the polymer (approximately 45-55° C.). High-pressure compaction ensures that the ODN is fully incorporated within the polymer lattice with a concomitant reduction in particle porosity; to minimize premature release of the ODN. Following compaction the matrix is again cryogenically ground in a Tekmar A-10 Analytical Mill (Glen Mills, Clifton, N.J.).

Size

Particles in the appropriate size range for engulfment by M-cells should be less than 10 microns, preferably less than 5 microns. Typically the range is between 5 nm to 50 microns, most preferably between 500 nm and 5 microns. These may be separated by sonic sifting through nickel mesh sieves with sieve openings of 5 or 10 microns. (Fisher Scientific, ATM Nos. L3M5 or L3M10 Pittsburgh, Pa.). Particle size distributions is determined by Particle Sizing Systems (Langhorne, Pa.). Distributions are determined on their Accusizer 780 Single Particle Optical Sizer, Particle Sizing System (Langhorne, Pa.) capable of measurements in the range 0.5 to 2500 microns.

Applications for Compositions

This system provides for oral controlled release of vaccine, which is both capable of protecting plasmid immunogens in the stomach and of providing optimized plasmid release in the colon by bioadherence. These immunogenic microparticles are, in turn, encapsulated into a secondary protective carrier, preferably an enteric carrier, such as soluble gelatin capsules coated with an acrylic resin soluble at a pH>7.0. The purpose of the secondary carrier is to protect the controlled release formulation as it passes through the stomach so that the plasmid/PLGA/adjuvant microparticles are made ready for delivery directly to the mucosal tissue of the colon.

Measurement of Bioadhesiveness

The experimental technique to measure bioadhesion is an adaptation of the method described by Chickering, et al., (J. Con. Rel. 34: 251-261 (1995)). Matrices, pressed as tablets with a 2.0 mm diameter, are suspended by a fine wire attached to one surface into a temperature-controlled cell containing a section of rat colon cut longitudinally to expose the lumen. The section is attached to the bottom of the cell and bathed in phosphate buffered saline adjusted to the pH of the colon. The wire, in turn, is suspended from the weighing arm of a Roller-Smith Precision Balance (Rosano Surface Tensiometer, Biolar Corporation, North Grafton, Mass.). This configuration allows the circular face of the tablet to be pressed into the mucosa with a force that can be varied up to the weight of the tablet less the buoyant force exerted by the medium on the tablet. After contact between the tablet face and mucosa for a predetermined time (1 minute), the tablet is slowly raised and the force required to break the contact is registered on the tensiometer scale. The adhesive force per unit area is given by:

F=(Δw)g/πr² g sec⁻² cm⁻¹=dyne cm⁻² Eq. 2

where

Δw=difference between tensiometer reading at rupture of the adhesive bond and the contact force (grams)

g=gravitational constant (980 cm sec⁻²)

r=tablet radius (cm)

This process eliminates the shear forces generated by emulsification during microsphere formation. First, a polymer foam of controlled pore size and density is prepared by lyophilization (freeze drying) of a polymer solution. A starting polymer concentration of 50 mg/ml for the lyophilization yields an open-celled foam with a density of approximately 70 mg/cm³ corresponding to a void volume of approximately 94 percent. The open-celled foam is then impregnated with an aqueous solution of the plasmid by a series of gentle evacuation/re-pressurization cycles. The impregnated foam is lyophilized to remove water and this procedure deposits the pDNA within the pores of the foam. Following vacuum drying of the foam, no further solvent is used except water in which the active agent is dissolved. The foam, immersed in a solution of the active agent, is impregnated with the solution by applying a vacuum to remove air from the foam and then repressurizing by admitting air, which forces the solution into the pores. This process is repeated several times. The solution loaded foam is then lyophilized to remove the water.

The plasmid-impregnated composite is then extruded under high pressure at a temperature not to exceed 55° C. The compacted matrix is then cryogenically ground and ultrasonically sieved to retain a particle size (as measured via microscopy) of less than five microns.

Plasmid/PLGA formulations are optimized for encapsulation efficiency and the rate at which the pDNA is released from the polymer particles. Control of the plasmid release is tuned by (1) the loading of the plasmid within the polymer, and (2) the pressure at which the plasmid/polymer matrix is extruded. At reduced loading concentrations, less of the vaccine is available at the polymer surface, alleviating problems involving the immediate loss of DNA upon exposure to aqueous media. Thus, more of the plasmid is retained within the polymer particles and the improved encapsulation efficiency reduces vaccine loss. In addition, loading within the polymer affects diffusion of the pDNA through the matrix. Control of encapsulation and release of the plasmid promotes protection of the plasmid within the polymer matrix from degradation enzymes and extends the period of time over which the pDNA is delivered. In addition, the magnitude of the early burst (roughly defined as that percentage of plasmid released within the first 24 hours) is directly related to the extrusion pressure.

II. Methods of Vaccination

The combination of mucosal priming with parenteral stimulation is a preferred method for delivering an antigen to develop the immune system. Recent findings support a method that combines a mucosal prime with controlled release parenteral stimulation. Therefore, in the preferred embodiment, a bioadhesive mucosal delivery system is used in concert with systemic immunization to develop long-lasting immune responses correlative to protective immunity. In the most preferred embodiment, “mucosal priming” is used in conjunction with parenteral immunization. As described herein, this system can be administered by oral and nasal administration, with a priming step, to induce mucosal immunity. Immunity has now been shown not just with protein antigens, but also with DNA vaccines, encoding the antigens.

This method of vaccination serves two purposes. The first is the controlled delivery of antigen such as protective ODNs to a mucosal site resulting in “priming” of mucosal receptors. The second is to augment this mucosal prime with parenteral stimulation. The priming of the mucosal system, accompanied by traditional vaccination, will result in an improved protection response.

A. Methods to Verify Antigen Release In Vitro

Verification of the biological activity of ODN incorporated into polymer/gelatin particles can be determined by ELISA. Release rates of ODN from the PLGA matrices can be measured in vitro. In a standard procedure, samples (e.g. 10-20 mg) are incubated in 1 ml volumes of phosphate buffered saline at 37° C. Replicate samples are centrifuged and ODN in the supernatant assayed. These measurements are done in triplicate. Release is monitored on days 1, 2, 3, 5, and 7, and weekly to six weeks.

For example, the release of a plasmid malaria vaccine (VR2578) from PLGA microparticles was characterized in vitro to assess plasmid encapsulation and expected delivery rate from the matrix. PLGA particles containing the encapsulated pDNA with an approximate mass of 10 mg were suspended in 1.5 mL of 0.1 M phosphate buffer saline (PBS). The suspension was incubated at 37° C. and shaken at 60 cycles per minute. A total of six samples were added to the water bath and the quantity of released plasmid was measured at times of 1, 4, and 24 h and at 7, 21, 28, 35, 42, 49, and 56 days. Upon removal from the water bath, suspended particles were isolated by centrifugation at 50,000 rpm for 10 min. The supernatant solution containing released pDNA was collected with a pipette.

The concentration of pDNA in solution was measured by UV spectroscopy as described by Tinsley-Brown et al. (2000). Approximately 0.5 mL of pDNA solution was added to a quartz cuvette of path length 1 cm and width of 0.2 cm. Solutions of native VR2578 were diluted in PBS to known concentrations to serve as calibration standards. These solutions with known concentrations of pDNA were used to measure the unknown concentrations of pDNA by creating an absorbance versus concentration standard curve. Absorbance was recorded at 260 nm for each solution on a Varian Cary Scan 100 UV/vis spectrophotometer. A reference absorbance background was provided by a PBS solution that was incubated with control PLGA particles not encapsulated with pDNA.

The quantity of plasmid encapsulated within the polymer particles was measured by accelerating the release of retained plasmid following 56 days of incubation. A basic environment to catalyze polymer degradation and vortex mixing to promote release of the plasmid from the polymer phase resulted in the remainder of encapsulated plasmid to be released. Tinsley-Brown et al. (2000) described this technique for measuring the quantity of plasmid loaded into PLGA systems. After 56 days of incubation, microparticles and the remaining encapsulated pDNA were isolated from the PBS supernatant. The microparticles were suspended in 1.5 mL of 0.2 M NaOH and incubated at 120° C. for 10 min. The basic environment and elevated temperatures promoted degradation of the biopolymer system and release of the pDNA. Following the incubation step, the suspended particles were agitated on a vortex mixer for 1 min. The concentration of VR2578 in solution was measured using UV spectroscopy. For concentration measurement in NaOH, solutions of known pDNA concentrations were created in 0.2 M NaOH for the calibration curve. In addition, the reference background was a NaOH solution incubated with control PLGA particles that did not contain any plasmid.

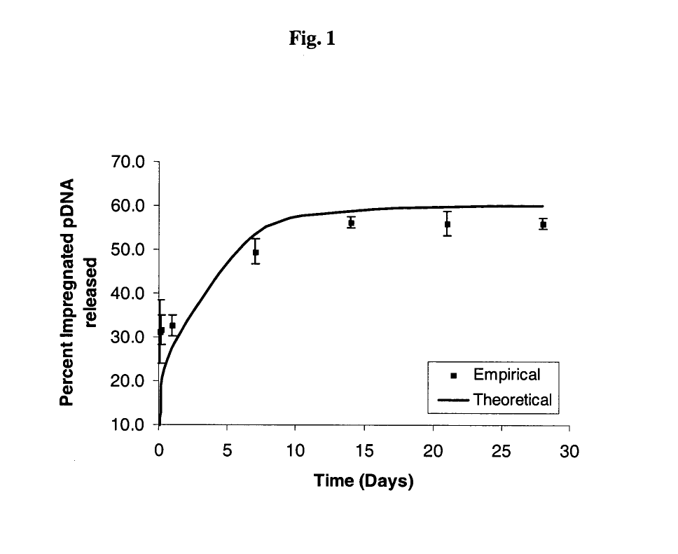

Release of the plasmid occurred at a controlled rate for 14 days from PLGA microparticles (see FIG. 1). The plasmid was effectively impregnated using the extrusion technique and the burst effect was significantly reduced. Approximately 30 percent of the plasmid was released immediately upon immersion of the particles into buffer. The remainder of the plasmid was retained within the particles. An additional 30 percent of the total plasmid impregnated within the microparticle system was released through 7 weeks with most of the released VR2578 detected after 14 days. Although the quantity of plasmid released significantly decreased between 14 and 21 days, released VR2578 was detected in the buffer environment through 49 days.

FIG. 1 shows theoretical values for the release of VR2578. These values were determined based on a model assuming Fickian diffusion of the plasmid to the buffer environment. For one-dimensional radial release from a sphere of a radius a, under perfect sink initial and boundary conditions, with a constant drug diffusion coefficient (D), Fick's second law may be written as:

$$\frac{\partial C}{\partial t} = D [ \frac{{\partial }^{2} C}{\partial {r}^{2}} + \frac{2}{r} \frac{\partial C}{\partial r} ] \\ where \\ t = 00 < r < a C = {C}_{1} \\ t > 0 r = a C = {C}_{0} \qquad Eq . 3$$

The solution to Fick's law under the specified conditions is (Crank 1975; Ritger 1987):

$$\frac{{M}_{t}}{{M}_{∞}} = 1 - \frac{6}{{π}^{2}} {\sum }_{n = 1}^{∞} \frac{1}{{n}^{2}} exp [ \frac{- D {n}^{2} {π}^{2} t}{{a}^{2}} ] \qquad Eq . 4$$

Using the empirical data collected in this study, the value of D calculates as 1.8×10⁻⁹ m²/s for early times. The theoretical release profile generally represented the empirical data (see FIG. 1). However, the model did not account for the immediate release of plasmid (“burst effect”) from the microparticle system.

B. Methods for In Vivo Evaluation of Immune Responses

The immune responses based on (1) nasal immunization, (2) injection, and (3) a combination of nasal/injection immunization is generally initially assessed in mice. Doses are determined based on review of the in vitro release profiles. Mice are tested for antibody response using the ELISA techniques. For example, separate groups of BALB/c mice (6-8 weeks of age) are immunized with 50 micrograms of ODN. Immunization groups include mice administered via parenteral, nasal, or (nasal+parenteral) routes. Control groups consists of nasal administered PLGA-only or PLGA with a control ODN. The in vivo evaluation methods are further described with reference to malaria vaccines, although it is understood the techniques are applicable to other types of vaccines.

Malaria

Dose response of malaria vaccines impregnated within PLGA particles is studied in mice using procedures established by Doolan et al., (J. Exper. Med. 183: 1739-1746 (1996)) and Sedegah, et al., (J. Immun. 164: 5905-5912 (2000)). BALB/c mice are administered the vaccine/PLGA particles via an intramuscular (IM) injection of the particles suspended in a medium consisting of 0.36 percent (w/v) sodium carboxy methyl cellulose, 3.6 percent (w/v) D-mannitol, and 0.07 percent (w/v) Tween 80 in distilled water. Another group is immunized with injections of naked pDNA in saline. Doses corresponding to 0.5, 5, and 50 μg of plasmid are administered via 50 μL injections in each tibialis anterior muscle. Mice are immunized at 0, 3, and 6 weeks with the vaccine/PLGA particles. In addition, groups of control mice receive injections of a plasmid/PLGA vehicle where the plasmid does not express proteins recognizing epitopes at the surface of malaria-affected cells and PLGA-only particles. Mice are immunized in groups of six specified by dose and vaccine formulation.

At weeks 5 and 8, animals are bled 200 to 300 μL from the tail vein with the blood to be tested for the presence of antibodies. An indirect fluorescent Ab test (IFAT) is used to detect serum levels of anti-Plasmodium yoelii sporozoite antibodies. Briefly, collected sera is incubated with air-dried sporozoites and antibody concentration is measured through binding of fluorescein isothiocyanate-labeled anti-mouse Ig as described by Sedegah et al., (Proc. Nat. Acad. Sci. USA 95: 7648-7653 (1998)).

Protective immunity of mice immunized with the pDNA/PLGA vaccines versus mice immunized with naked pDNA is verified by monitoring cytotoxic T lymphocyte (CTL) and gamma interferon (IFN-γ) response. CTL activity is studied using a ⁵¹Cr release assay conducted on spleen cells harvested from immunized mice. Spleen cells are incubated in vitro with ⁵¹Cr-labeled target cells containing the epitope of interest. The net percent specific lysis is calculated based upon the percent of positive lysis target cells minus the percent of negative lysis controls (Sedegah, et al., J. Immun. 164: 5905-5912 (2000)). IFN-γ response is also found by incubation of spleen cells with target cells containing the epitope of interest. Levels of IFN-γ are found by the addition of anti-mouse IFN-γ antibody. The numbers of IFN-γ-spot forming cells are counted per million spleen cells (Sedegah, et al., Proc. Nat. Acad. Sci. USA 95: 7648-7653 (1998)). Protective immunity is established by demonstration of both CTL and IFN-γ activity.

Following the dose response study in mice, the pDNA/PLGA vaccine system is tested and challenged in primates. Malaria-naive rhesus monkeys are randomized into groups of three for each vaccine/PLGA formulation based upon the procedure outline by Wang, et al. (Infect. Immun. 66(9): 41934202 (1998)). Control groups receive injections of naked pDNA in saline and a plasmid/PLGA formulation that does not express for proteins of sporozoite infected cells. Immunizations are conducted at 0, 4, and 8 weeks via administration of pDNA/PLGA suspensions. Injections consist of 1 mL total volume delivered IM amongst four sites: triceps, tibialis anterior, deltoid, and quadriceps. Blood samples are collected at 2 and 4 weeks post-immunization corresponding to weeks 6, 8, 10, and 12.

Anthrax

The anthrax vaccine is generally administered as an oral dose form and delivers the DNA plasmids encoding PA and LF antigens of the anthrax vaccine to the colon where attachment to the M-cells is facilitated by the bioadhesive properties of the PA formulation. The vaccine stimulates three distinct antibody peaks. (See Gu, et al., Vaccine 17: 340-344 (1999); Price, et al., Infec. Immun. 69(7): 4509-4515 (2001)).

Modifications and variations of the methods and reagents described herein will be obvious to those skilled in the art and are intended to come within the scope of the appended claims.

We claim:

1. A vaccine composition for inducing an immune response to a pathogen comprising a nucleic acid encoding an antigen eliciting an immune response to the pathogen encapsulated in a mucoadhesive controlled release particulate formulation.

2. The composition of claim 1 wherein the formulation comprises a biodegradable polymer.

3. The composition of claim 2 further comprising a mucoadhesive polymer coating.

4. The composition of claim 1 further comprising an enteric outer coating or capsule.

5. The composition of claim 1 having a particulate diameter of less than five microns.

6. The composition of claim 2 formed by
lyophilizing a solution of a biodegradable polymer to form an open-celled polymeric foam of approximately 95% void volume,
impregnating the foam with an aqueous solution of the nucleic acid,
lyophilizing the foam to remove the water, and
extruding the resulting matrix at ultrahigh pressures.

7. The composition of claim 2 wherein the method further comprises
cryogenically grinding the matrix to an average particle size of fifteen microns in diameter; and
sieving to isolate particles less than five microns in diameter.

8. The composition of claim 1 wherein the polymer is a low molecular weight poly(D,L-lactide-co-glycolide).

9. The composition of claim 1 wherein the pathogen is selected from the group consisting of malaria, tularemia, anthrax, and H. pylori.

10. The composition of claim 1 further comprising providing an adjuvant with the antigen.

11. The composition of claim 1 wherein the antigen is expressed or released for a period of weeks to months.

12. A porous particulate formulation comprising an antigen and having a mucoadhesive coating, wherein the formulation is suitable for administration orally or nasally.

13. The formulation of claim 12 wherein the antigen is selected from the group consisting of a malaria antigen, a tularemia antigen, an anthrax antigen, and a H. pylori antigen.

14. The formulation of claim 12 wherein the antigen is a peptide.

15. The formulation of claim 12 wherein the antigen is expressed from nucleic acid incorporated into the particulate formulation.

16. The formulation of claim 12 further comprising an adjuvant.

17. The formulation of claim 12 wherein the particulate has a mucoadhesive coating and a diameter of less than five microns.

18. The formulation of claim 12 wherein the formulation is enterically coated or encapsulated within an enteric capsule.

19. The formulation of claim 12 wherein the antigen is expressed or released for a period of weeks to months.

20. A method of inducing an immune response to a pathogen comprising administering to a patient by an oral or nasal route a vaccine composition comprising a nucleic acid encoding an antigen eliciting an immune response to the pathogen encapsulated in a mucoadhesive controlled release particulate formulation.

21. The method of claim 20 wherein a priming dose is administered before an immunizing dose is administered.
